(19) United States
(12) Patent Application Publication
(10) Pub. No.: US 2014/0378496 A1
(43) Pub. Date: Dec. 25, 2014

(54) OPIOID ANALGESICS AND 3-HYDROXYACETANILIDE FOR TREATING PAIN

(71) Applicant: SLX PHARMACEUTICALS, LLC, San Francisco, CA (US)

(72) Inventor: Jonathan Wray, San Francisco, CA (US)

(21) Appl. No.: 14/357,731

(22) PCT Filed: Nov. 9, 2012
(86) PCT No.: PCT/US2012/064427

§ 371 Date: May 12, 2014

Related U.S. Application Data
(60) Provisional application No. 61/558,436, filed on Nov. 11, 2011.
(60) Provisional application No. 61/719,104, filed on Oct. 26, 2012.

(51) Int. Cl. A61K 31/485; A61K 31/167

(52) U.S. Cl. CPC A61K 31/485; A61K 31/167
USPC 514/282

(57) ABSTRACT

Pharmaceutical combinations of opioid analgesics and analgesics that act through non-opioid mechanisms are commonly used to provide pain relief. An example of this pharmaceutical combination is the product Vicodin™, where the opioid analgesic is hydrocodone and the non-opioid is acetaminophen. However, liver toxicity from the acetaminophen component is common. The invention provides an improvement over the opioid and acetaminophen pharmaceutical combinations for the management of pain by the concomitant administration of an opioid analgesics and the non-opioid analgesic 3-hydroxyacetanilide. This combination has been found to exhibit unexpectedly enhanced analgesic activity when dosed orally in a mammal.

### BACKGROUND OF THE INVENTION

Opioid analgesics have been used for the relief of moderate to severe pain. Severe pain, particularly, has required the use of opioid analgesics in large and increasing dosage amounts. A disadvantage of the opioid analgesic is the development of dependence or addiction and tolerance to their action. Further, adverse reactions to large doses are respiratory and circulatory depression.

Pharmaceutical combinations of opioid analgesics and analgesics that act through non-opioid mechanisms (e.g. non-steroidal anti-inflammatory drugs (NSAID)) are commonly used to provide pain relief. An example of this pharmaceutical combination is the product Vicodin™, where the opioid analgesic is hydrocodone and the non-opioid is acetaminophen.

Due to the widespread prescribing of these opioid/acetaminophen combination products, and the prevalence of over-the-counter products containing acetaminophen, liver toxicity from high doses of acetaminophen is common. In 2011, in response to reports of liver toxicity, The United States Food and Drug Administration (FDA) asked manufacturers of prescription combination products that contain acetaminophen to limit the amount of acetaminophen to no more than 325 milligrams (mg) in each tablet or capsule. The FDA is also requiring manufacturers to update labels of all prescription combination acetaminophen products to warn of the potential risk for severe liver injury.

The mechanism by which acetaminophen exerts its analgesic effects is not entirely known, but the following mechanisms have been postulated to explain acetaminophen-induced analgesia: selective inhibition of cyclooxygenase activity in the central nervous system, interaction with spinal 5-HT3 receptors, interference with spinal substance P receptors or inhibition of neurons excited by substance P, activation of supraspinal descending inhibitory pathways, increase in pituitary β-endorphin secretion, direct effects of neuronal membrane potentials.

3-Hydroxyacetanilide (CAS Registry Number 621-42-1), a region-isomer of acetaminophen, appears in many patent documents, including: U.S. Pat. Nos. 8,158,682; 6,759,064; 6,515,081; 6,492,428; 6,399,199; 6,242,646; 5,461,075; 5,136,868; 5,099,030; 5,075,488; 5,045,565; 5,026,731; 5,013,759; 4,906,286; 4,898,887; 4,874,866; 4,831,136; 4,812,446; 4,772,715; 4,766,233; 4,709,052; 4,681,897; 4,677,202; 4,661,438; 4,607,000; 4,599,342; 4,581,330; 4,576,905; 4,564,633; 4,544,669; 4,544,668; 4,532,139; 4,493,848 4,424,205; 4,401,663; 4,238,538; 20120230916; 20100292755; 20100290998; 20100234470; 20100129854; 20090118242; 20090117167; 20090111792; 20090062359; 20090054527; 20080262091; 20080260791; 20070036876; 20060269628; 20060148903; 20050058734; 20050049229; 20050020690; 20050019436; 20040191338; 20040186182; 20040161481; 20040156931; 20030072821; and 20020007022. U.S. Pat. No. 4,238,508 further describes the analgesic effects of 3-hydroxyacetanilide when dosed interpeirtonerally in mice. However, its biological target(s) and mechanism by which it exerts its analgesic effects are unknown.

As mentioned, 3-hydroxyacetanilide is a regio-isomer of acetaminophen which has the chemical name 4-hydroxyacetanilide. Despite their structural similarity, the two molecules demonstrate greatly different activities in regard to liver toxicity (hepatotoxicity), and 3-hydroxyacteanilide is often reported as the non-hepatotoxic isomer of acetaminophen. Studies of the metabolism of acetaminophen have shown that its hepatotoxicity is associated with glutathione depletion, likely caused by glutathione conjugation to a reactive metabolite of acetaminophen. 3-Hydroxyacetanilide can also be metabolized to a similar reactive intermediate however; its metabolism leads to significantly less glutathione depletion and reduced hepatotoxicity. The divergent toxicities of these isomers is instructive in that, just as it is difficult to predict toxicity of structurally similar compounds it is also difficult to predict the nature of drug-drug interactions in a biological system.

When two drugs which produce similar physiological effects such as antinociception are administered together, certain questions arise for example: How does an effect of the combination compare with the effects of the individual drugs given at the same dose. Is the observed effect of the combination greater (or less) than the expected effect? Even the administration of a single drug places it in contact with a myriad of biological circumstances that may affect its activity and efficacy.

Opioids are often used in combination with other analgesics for the management of pain. However these interactions (sub-additive, additive, or super-additive (synergistic)) have been shown to be dependent on the particular experimental conditions and pharmacological model. In a model of chronic neuropathic pain, systemic ketorolac or piroxicam (analgesics of the NSAIDS class) was found to synergize with spinal morphine. Interestingly, these same authors failed to identify synergy between ketorolac and morphine using a thermal pain model.

In spite of the above reports, there is currently a need for improved compositions and methods for treating pain. In particular, there is a need for new therapeutic combinations of opioid analgesics and non-opioid analgesics that produce fewer side effects (e.g. liver toxicity) and/or that provide acceptable pain relief using lower quantities of opioid.

### SUMMARY OF THE INVENTION

It has been found that combinations of opioid analgesics and 3-hydroxyacetanilide provide a synergistic analgesic effect when administered orally to a mammal. Accordingly, the invention provides new therapeutic combinations of opioid analgesics and 3-hydroxyacetanilide, as well as methods for using such combinations to treat pain in animals (e.g. humans). The compositions and methods of the invention provide pain relief while producing fewer side effects (e.g. liver toxicity). Due to the synergistic effects produced by the compositions and methods of the invention, effective pain relief can be achieved using lower quantities of opioid.

The invention provides a pharmaceutical composition comprising an analgesic amount of an opioid analgesic and/or at least one pharmaceutically acceptable salt thereof, and 3-hydroxyacetanilide.

The invention provides a pharmaceutical composition comprising an opioid analgesic or a pharmaceutically acceptable salt thereof, 3-hydroxyacetanilide, and a pharmaceutically acceptable carrier.

The invention also provides a pharmaceutical composition comprising up to about 20 mg of hydrocodone, 3-hydroxyacetanilide, and a pharmaceutically acceptable carrier.

The invention also provides a pharmaceutical composition comprising up to about 15 mg of hydrocodone, 3-hydroxyacetanilide, and a pharmaceutically acceptable carrier.

The invention also provides a pharmaceutical composition comprising up to about 10 mg of hydrocodone, 3-hydroxyacetanilide, and a pharmaceutically acceptable carrier.

The invention provides a method of treating pain in humans and other mammals by the systemic administration of analgesic amounts of an opioid analgesics, the improvement comprising the step of the concomitant systemic administration of an analgesic amount of 3-hydroxyacetanilide.

The invention also provides a method for treating pain in humans and other mammals comprising the concomitant systemic administration of an opioid analgesics, and an analgesic amount of 3-hydroxyacetanilide.

The invention provides a method for treating pain in an animal (e.g. a human), comprising administering an opioid analgesic or a pharmaceutically acceptable salt thereof to the animal, and orally administering 3-hydroxyacetanilide to the animal.

The invention provides a method for treating pain in a human comprising administering an opioid analgesic or a pharmaceutically acceptable salt thereof, and 3-hydroxyacetanilide to the human.

The invention provides an opioid analgesic for the treatment of pain when administered with 3-hydroxyacetanilide.

The invention provides 3-hydroxyacetanilide for the treatment of pain when administered with an opioid analgesic.

The invention provides an opioid analgesic for the treatment of pain when administered with orally administered 3-hydroxyacetanilide.

The invention provides orally administered 3-hydroxyacetanilide for the treatment of pain when administered with an opioid analgesic.

The invention also provides the use of 3-hydroxyacetanilide to prepare a medicament for treating pain in an animal when administered with an opioid analgesic. In one embodiment the medicament is suitable for oral administration.

The invention also provides the use of an opioid analgesic to prepare a medicament for treating pain in an animal when administered with 3-hydroxyacetanilide.

The invention also provides the use of an opioid analgesic and 3-hydroxyacetanilide to prepare a medicament for treating pain in an animal. In one embodiment the medicament is suitable for oral administration.

### BRIEF DESCRIPTION OF THE FIGURES

FIG. 1 shows the structures of hydrocodone and 3-hydroxyacetanilide.

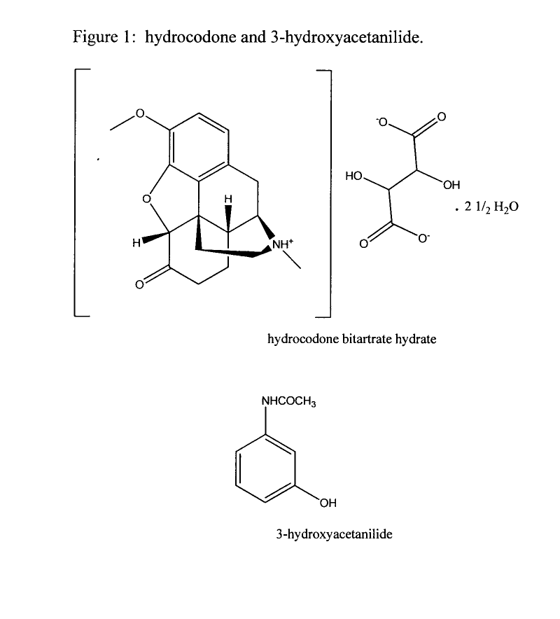

FIG. 2 shows the structures of oxyocodone hydrochloride and 3-hydroxyacetanilide.

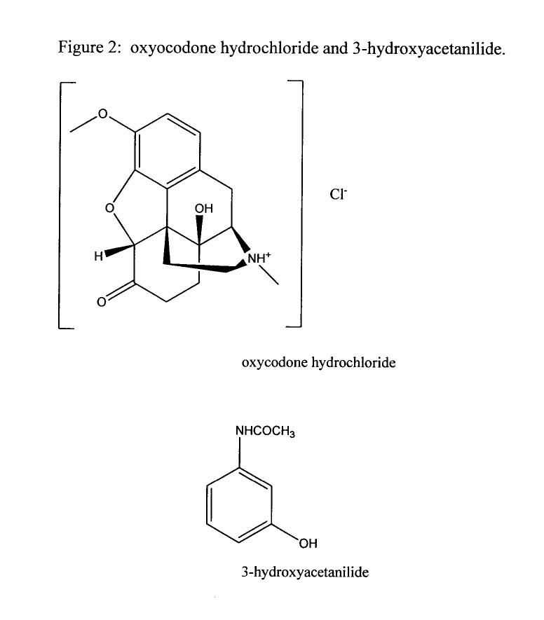

FIG. 3 shows the structures of codeine hydrochloride and 3-hydroxyacetanilide.

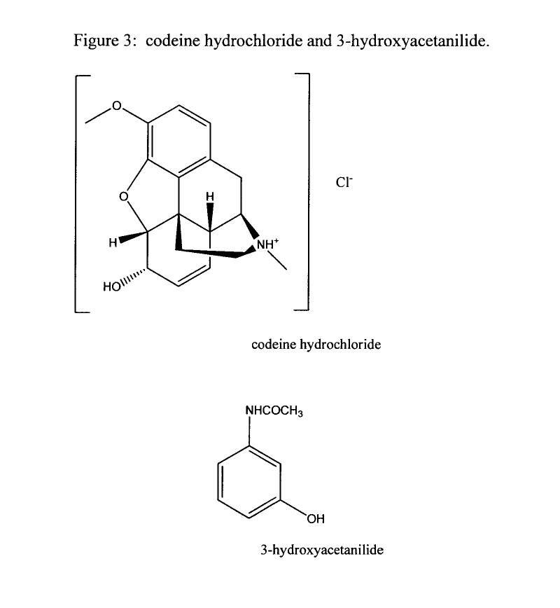

FIG. 4 shows an ester prodrug of hydrocodone and 3-hydroxyacetanilide.

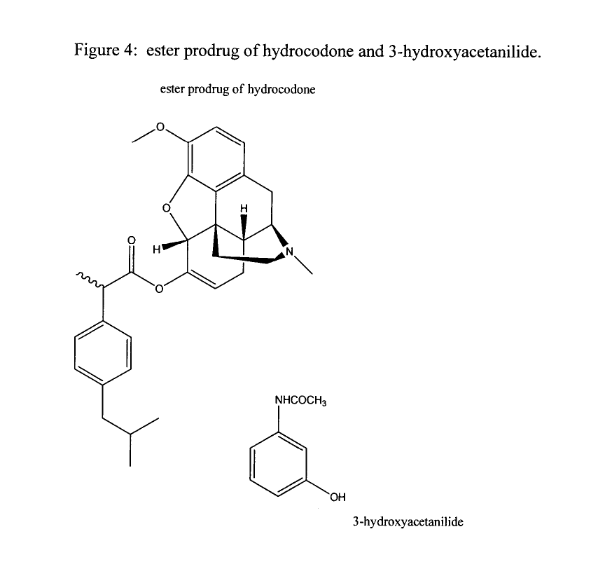

FIG. 5 shows an N-17-alkylated prodrug of hydrocodone and 3-hydroxyacetanilide.

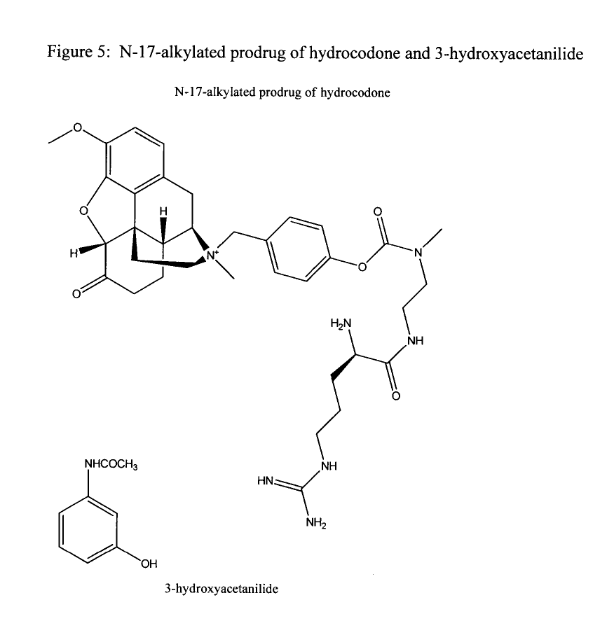

FIG. 6 shows a ketone-modified opioid prodrug of hydrocodone and 3-hydroxyacetanilide

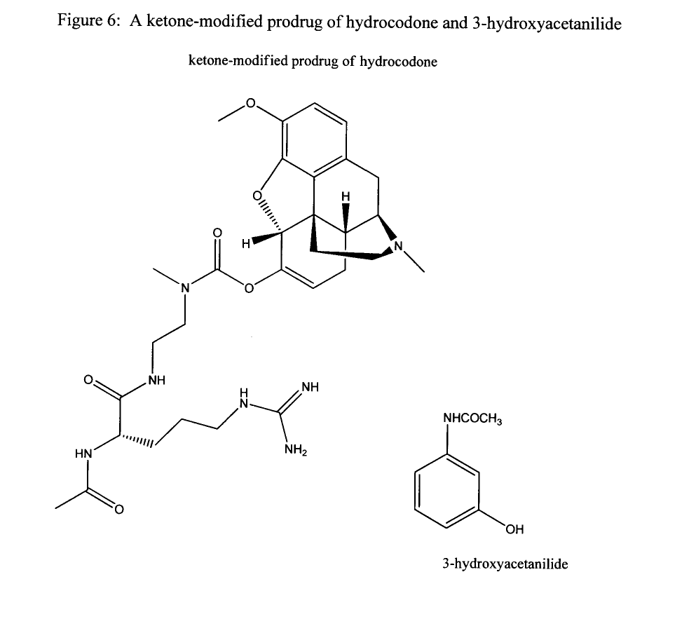

FIG. 7 shows linear regression analysis data (effect vs log dose) from the Examples below.

FIG. 8 shows linear regression analysis (effect vs dose) from the Examples below.

### DETAILED DESCRIPTION OF THE INVENTION

Opioid analgesics are well known and have been used for many years for the treatment of moderate to severe pain. The term opioid analgesic when used herein includes but is not limited to hydrocodone, oxycodone, and codeine. Hydrocodone, oxycodone, and codeine are preferred because they are the opioid analgesics commonly combined with acetaminophen in combination analgesic products.

The Controlled Substance Act (CSA), enacted into law by the Congress of the United States as Title II of the Comprehensive Drug Abuse Prevention and Control Act of 1970 scheduled hydrocodone, oxycodone, and codeine as Schedule III narcotics when combined at certain doses with non-narcotic analgesics (e.g. aspirin, ibuprofen), but the more strictly regulated as Schedule II in stand-alone dosage units.

The use of “opioid” is meant to include any drug that activates the opioid receptors found in the brain, spinal cord and gut. There are four broad classes of opioids: naturally occurring opium alkaloids, such as morphine (the prototypical opioid), codeine and thebaine; endogenous opioid peptides; semi-synthetics such as heroine, oxycodone and hydrocodone that are produced by modifying natural opium alkaloids (opiates) and have similar chemical structures; and pure synthetics such as fentanyl and methadone that are not produced from opium and may have very different chemical structures than the opium alkaloids. Additional examples of opioids are hydromorphone, oxymorphone, levorphanol, dihydrocodeine, meperidine, diphenoxylate, sufentanil, alfentanil, propoxyphene, pentazocine, nalbuphine, butorphanol, buprenorphine, meptazinol, dezocine, and pharmaceutically acceptable salts thereof.

Hydrocodone is a narcotic analgesic, which acts as a weak agonist at opioid receptors in the central nervous system (CNS). It primarily affects the (mu) receptor (OP3), but also exhibits agonist activity at the delta receptor (OP1) and kappa receptor (OP2). Additionally, hydrocodone displays antitussive properties by suppressing the cough reflex in the medullary cough center of the brain. The use of “hydrocodone” is meant to include a semisynthetic narcotic analgesic and antitussive derived from either codeine or thebaine with multiple actions qualitatively similar to those of codeine. It is commonly used for the relief of moderate to moderately severe pain. Other salt forms of hydrocodone, such as hydrocodone bitartrate and hydrocodone polistirex, are encompassed by the present invention.

Hydrocodone is used for the treatment of moderate to moderately severe pain and for inhibition of cough (especially dry, nonproductive cough). The prodrugs of hydrocodone may be administered for the relief of pain or for cough depression or for the treatment of any condition that may require the blocking of opioid receptors.

Hydrocodone produgs and other prodrugs of opioids may provide reduced potential for overdose, reduced potential for abuse or addiction and/or improve hydrocodone's characteristics with regard to high toxicities or suboptimal release profiles. Without wishing to be limited by theory, it is believed that overdose protection may occur due to the prodrug's being exposed to different enzymes and/or metabolic pathways by oral administration where the conjugate is exposed through the gut and first-pass metabolism as opposed to the exposure to enzymes in the circulation or mucosal membranes which limits the ability of hydrocodone to be released from the pro-drug. Therefore, abuse resistance is provided by limiting the “rush” or “high” available from the active hydrocodone released by the prodrug and limiting the effectiveness of alternative routes of administration. Hydrocodone prodrugs of this type preferably have no or a substantially decreased pharmacological activity when administered through injection or intranasal routes of administration. However, they remain orally bioavailable.

When the terms opioid analgesic or 3-hydroxyacetanilide are used herein, it is to be understood that any of the pharmaceutically suitable salts thereof which have analgesic properties in man and other mammals are included by the term. Such salts include the hydrochlorides, hydrobromides, hydroiodides, sulfates, bisulfates, nitrates, citrates, tartrates, bitartrates, phosphates, malates, maleates, fumarates, succinates, acetates, terephthalates, pamoates, aluminum, calcium, potassium, and sodium.

In one embodiment the invention provides a combination of chemical compounds useful in the management of pain in a mammal. The combination is an opioid analgesic and 3-hydroxyacetanilide. Certain specific opioid analgesics include hydrocodone, oxycodone, and codeine.

In one embodiment of the invention the opioid analgesic is selected from the group consisting of oxycodone or hydrocodone, or a pharmacologically acceptable salt, thereof.

In one embodiment of the invention the opioid analgesic is codeine, or a pharmacologically acceptable salt, thereof.

In one embodiment of the invention the opioid analgesic is hydrocodone, or a pharmacologically acceptable salt, thereof at a dose per 100 ml (i.e., a liquid), which has no more than 300 mg of (dissolved) hydrocodone in addition to the therapeutic amount of 3-hydroxacetanilide.

In one embodiment of the invention the opioid analgesic is hydrocodone, or a pharmacologically acceptable salt, thereof at a dose per dosage unit (i.e., a solid, pill or capsule), which has no more than 15 mg of hydrocodone in addition to the therapeutic amount of 3-hydroxacetanilide.

In one embodiment of the invention the opioid analgesic is oxycodone, or a pharmacologically acceptable salt, thereof at a dose per dosage unit (i.e., a solid, pill or capsule), which has no more than 10 mg of oxycodone in addition to the therapeutic amount of 3-hydroxacetanilide.

In one embodiment of the invention the opioid analgesic is codeine, or a pharmacologically acceptable salt, thereof, at a dose per dosage unit (i.e., a solid, pill or capsule), which has no more than 60 mg of codeine in addition to the therapeutic amount of 3-hydroxacetanilide.

In one embodiment of the invention the opioid analgesic is selected from the group consisting of a prodrug of oxycodone or a prodrug of hydrocodone, or a pharmacologically acceptable salt, thereof.

The opioid analgesic and 3-hydroxyacetanilide can be administered in the same dosage unit or can be prepared in separate dosage units and the dosage units administered at the same time. Different forms of dosage units can be used (i.e., a tablet of 3-hydroxyacetanilide, and an injection of opioid).

The compositions of the present invention are preferably presented for systemic administration to humans and animals in unit dosage forms, such as tablets, capsules, pills, powders, granules, suppositories, sterile parenteral solutions or suspensions, sterile non-parenteral solutions or suspensions, and oral solutions or suspensions and the like, containing suitable quantities of the combination of active ingredients.

The efficacious doses used for treating pain in a human for of the combination of an opioid with 3-hydroxyacetanilide; either administered in the same dosage unit, or in separate dosage units administered at the same time, can be determined by a physician using known means, taking into consideration that the demonstrated synergistic action of the combination may allow for a lower dose of the both compounds in the combination compared to their individual doses if they were to be dosed alone. The demonstrated synergistic action could lower the opioid dose by as much as 50% (e.g. from 20 mg to 10 mg of hydrocodone) in the combination product compared to the dose of the opioid administered alone.

For oral administration either solid or fluid unit dosage forms can be prepared.

Powders are prepared quite simply by comminuting the active ingredients to a suitably fine size and mixing with a similarly comminuted diluent. The diluent can be an edible carbohydrate material such as lactose or starch. Advantageously, a sweetening agent or sugar is present as well as a flavoring oil.

Capsules are produced by preparing a powder mixture and filling into formed gelatin sheaths. Advantageously, as an adjuvant to the filling operation, lubricant such as talc magnesium stearate, calcium stearate and the like is added to the powder mixture before the filling operation.

Soft gelatin capsules are prepared by machine encapsulation of a slurry of active ingredients with an acceptable vegetable oil, light liquid petrolatum or other inert oil or triglyceride.

Tablets are made by preparing a powder mixture, granulating or slugging, adding a lubricant and pressing into tablets. The powder mixture is prepared by mixing the active ingredients, suitably comminuted, with a diluent or base such as starch lactose, kaolin, dicalcium phosphate and the like. The powder mixture can be granulated by wetting with a binder such as corn syrup, gelating solution, methylcellulose solution or acacia mucilage and forcing through a screen. As an alternative to granulating, the powder mixture can be slugged, i.e., run through the tablet machine and the resulting imperfectly formed tablets broken into pieces (slugs). The slugs can be lubricated to prevent sticking to the tablet-forming dies by means of the addition of stearic acid, a stearate salt, talc or mineral oil. The lubricated mixture is then compressed into tablets.

Advantageously, the tablet can be provided with a protective coating consisting of a sealing coat or enteric coat of shellac, a coating of sugar and methylcellulose and a polish coating of carnauba wax.

Fluid unit dosage forms for oral administration such as syrups, elixirs and suspensions can be prepared wherein each teaspoonful of composition contains a predetermined amount of the active ingredients for administration. The water-soluble forms can be dissolved in an aqueous vehicle together with sugar, flavoring agents and preservatives to form a syrup. An elixir is prepared by using a hydroalcoholic vehicle with suitable sweeteners together with a flavoring agent. Suspensions can be prepared of the insoluble forms with a suitable vehicle with the aid of a suspending agent such as acacia, tragacanth, methylcellulose and the like.

For parenteral administration, fluid unit dosage forms are prepared utilizing the active ingredients and a sterile vehicle, water being preferred. The active ingredients, depending on the form and concentration used, can be either suspended or dissolved in the vehicle. In preparing solutions the water-soluble active ingredients can be dissolved in water for injection and filter sterilized before filling into a suitable vial or ampule and sealing. Advantageously, adjuvants such as a local anesthetic, preservative and buffering agents can be dissolved in the vehicle. Cosolvents such as ethanol or propylene glycol can be used in the solvent system. Parenteral suspensions are prepared in substantially the same manner except that the active ingredients are suspended in the vehicle instead of being dissolved and sterilization cannot be accomplished by filtration. The active ingredients can be sterilized by exposure to ethylene oxide before suspending in the sterile vehicle. Advantageously, a surfactant or wetting agent is included in the composition to facilitate uniform distribution of the active ingredients.

In addition to oral and parenteral administration, the rectal and vaginal routes can be utilized. Active ingredients can be administered by means of a suppository. A vehicle which has a melting point at about body temperature or one that is readily soluble can be utilized. For example, cocoa butter and various polyethylene glycols (carbowaxes) can serve as the vehicle.

The term “unit dosage form” as used in the specification and claims refers to physically discrete units suitable as unitary dosages for human and animal subjects, each unit containing a predetermined quantity of active material calculated to produce the desired therapeutic effect in association with the required pharmaceutical diluent, carrier or vehicle. The specification for the novel unit dosage forms of this invention are dictated by and are directly dependent on (a) the unique characteristics of the active material and the particular therapeutic effect to be achieved, and (b) the limitation inherent in the art of compounding such an active material for therapeutic use in humans, as disclosed in this specification, these being features of the present invention. Examples of suitable unit dosages forms in accord with this invention are tablets, capsules, troches, suppositories, powder packets, wafers, cachets, teaspoonfuls, tablespoonfuls, dropperfuls, ampules, vials, segregated multiples of any of the foregoing and other forms as herein described.

The following example is illustrative of the present invention, but is not intended to be limiting.

### EXAMPLE 1

In order to test the analgesic activity of the combination of an opioid (hydrocodone) and 3-hydroxyacetanilide, both compounds were tested separately and in combination in a visceral pain model in mice.

### MATERIALS AND METHODS

Experimental animals: One hundred fifty-five 4-week old, male Swiss Webster mice (stock number 024) were purchased from Charles River Laboratories (Portage, Mich.). The study animals were allowed an acclimation period of 1-2 weeks prior to dose initiation. The mice were housed 4-6 per cage and maintained in the Innovive caging system (San Diego, Calif.) upon arrival at. In accordance with the Guide for Care and Use of Laboratory Animals (Eighth Edition), mouse rooms were maintained at temperatures of 66-75 degrees Fahrenheit and relative humidity between 30% and 70%. Cages were monitored daily to ensure the Innovive system maintained 50 air changes per hour and positive pressure. The rooms were lit by artificial light for 12 hours each day (7:00 AM -7:00 PM). Animals had free access to distilled water and Teklad Global Rodent Diet 2018 (Harlan Laboratories, Madison, Wis.) for the duration of the study except during the experiment when food and water access was withheld.
Vehicle and Compound formulations: The vehicle (0.5% carboxy methyl cellulose, CMC) was formulated on-site weekly for the duration of the study, stored at 4° C., and allowed to come to room temperature prior to formulation every day. Carboxy methyl cellulose was provided by the client from Sigma Aldrich (catalog number C9481). The test articles and acetic acid (0.6%) were formulated on-site daily for the duration of the study. 3-hydroxyacetanilde and Hydrocodone (+)-bitartrate were purchased from Sigma Aldrich (catalog numbers A7205 & H4516, St. Louis, Mo.) and acetic acid was purchased from Thermo Fisher Scientific (Acros #42322-5000, Pittsburgh, Pa.). Compound solutions were sonicated and vortexed as needed prior to dosing to create a homogenous solution.
Study design: Vehicle and test articles were administered via oral gavage (PO) in dosing volumes of 10 ml/kg at T=−45 minutes (min) on Day 1 of the study. At T=0 min, acetic acid was administered via intraperitoneal (IP) injection at a dosing volume of 10 ml/kg. The writhing protocol (abdominal contortion test) was subsequently carried out on each animal. Animals were euthanized immediately following the writhing protocol on Day 1. The study design and treatment groups for all mice are represented in Table 1.
Abdominal contortion procedure (Writhing protocol): A writhe was characterized by a wave of contraction of the abdominal musculature followed by the extension of the hind limbs. Every day of the procedure began with a vehicle-treated animal for baseline measurements. During the first 10 days of the study, each day contained animals from Groups 2-11. During the last 4 days of the study, each day contained animals from Groups 12-15.

Animals were allowed to acclimate to the testing room for a minimum of 1 hour prior to the experiment. Immediately following administration of vehicle or test compounds (T=−45 min), animals were placed into individual clear, plexiglass cylindrical holders for the duration of the experiment. Therefore, animals were acclimated to the holders for 45 minutes prior to acetic acid administration. Acetic acid was administered at T=0 min, which began the writhing protocol experiment. The number of writhes over a 5 min duration were counted (5-10 and 15-20 min post-dose) starting 5 min after acetic acid administration.

TABLE 1
Study design

| | | Treatment (10 ml/kg) | | | |
|---|---|---|---|---|---|
| | | | Dose | | Conc. |
| Group | Number | Test article | (mg/kg) | Route | (mg/mL) |
| 1 | 14 | vehicle | 0 | PO | 0 |
| 2 | 10 | hydrocodone | 2 | PO | 0.2 |
| 3 | 10 | hydrocodone | 5 | PO | 0.5 |
| 4 | 10 | hydrocodone | 10 | PO | 1.0 |
| 5 | 10 | hydrocodone | 20 | PO | 2.0 |
| 6 | 10 | hydrocodone | 30 | PO | 3.0 |
| 7 | 10 | 3-hydroxyacetanilide | 50 | PO | 5.0 |
| 8 | 10 | 3-hydroxyacetanilide | 100 | PO | 10 |
| 9 | 10 | 3-hydroxyacetanilide | 200 | PO | 20 |
| 10 | 10 | 3-hydroxyacetanilide | 300 | PO | 30 |
| 11 | 10 | 3-hydroxyacetanilide | 400 | PO | 40 |
| 12 | 10 | 3-hydroxyacetanilide + | 50 + 1 | PO | 5 + 0.1 |
| | | hydrocodone | | | |
| 13 | 10 | 3-hydroxyacetanilide + | 100 + 2 | PO | 10 + 0.2 |
| | | hydrocodone | | | |
| 14 | 10 | 3-hydroxyacetanilide + | 200 + 4 | PO | 20 + 0.4 |
| | | hydrocodone | | | |
| 15 | 10 | 3-hydroxyacetanilide + | 300 + 6 | PO | 30 + 0.6 |
| | | hydrocodone | | | |

Statistical analysis: All treatment groups started the study with N=10 mice. The data were reviewed for identification and elimination of non-responders. A non-responder was defined as an animal that did not appear to respond to the acetic acid injection (i.e., no apparent abdominal contortions) in reference to the Vehicle Control animal assessed at the beginning of each experimental day. In addition, during the calculation of the ED₅₀ concentrations as well as during the final data review, any negative percents calculated for the percent antinociceptive activity (% AA) were changed to 0%. After Groups 2-11 measurements were completed, statistical calculations for the ED₅₀ concentrations of hydrocodone and 3-hydroxyacetanilide were calculated in Prism 5.0d (GraphPad Software) using the percent antinociceptive activity (% AA) and the log of each dose. Because 3-hydroxyacetanilde's dose response curve was bell-shaped, the lowest dose (50 mpk) was removed from further analysis, as it appeared to be an outlier. Outliers were screened by testing the group's mean versus the standard error of the mean (SEM) for said time point. If the relationship of SEM to mean was in excess of 10%, then the data points of that group at that time point were carried through an outlier test. Data points outside a z-score variation of 3.0 were listed as outliers and not included in the mean or SEM for the group. No statistical outliers were found in the study data after removal of non-responders.

TABLE 2
Results:

| Test Article | Dose (mg/kg) | % AA (mean/SEM) |
|---|---|---|
| hydrocodone | 2 | 28.03/9.72 |
| hydrocodone | 5 | 52.73/9.23 |
| hydrocodone | 10 | 77.24/9.23 |
| hydrocodone | 20 | 81.20/5.62 |
| hydrocodone | 30 | 96.98/1.62 |
| 3-hydroxyacetanilide | 100 | 17.10/7.46 |
| 3-hydroxyacetanilide | 200 | 20.05/7.71 |
| 3-hydroxyacetanilide | 300 | 24.00/7.00 |
| 3-hydroxyacetanilide | 400 | 35.42/10.33 |
| 3-hydroxyacetanilide + | 50 + 1 | 17.03/6.51 |
| hydrocodone | | |
| 3-hydroxyacetanilide + | 100 + 2 | 51.73/11.62 |
| hydrocodone | | |
| 3-hydroxyacetanilide + | 200 + 4 | 58.11/9.97 |
| hydrocodone | | |
| 3-hydroxyacetanilide + | 300 + 6 | 85.23/5.46 |
| hydrocodone | | |

### Linear Regression Analysis and Combination Analysis:

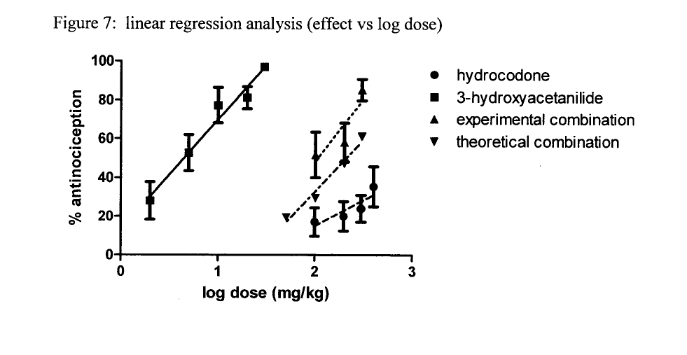

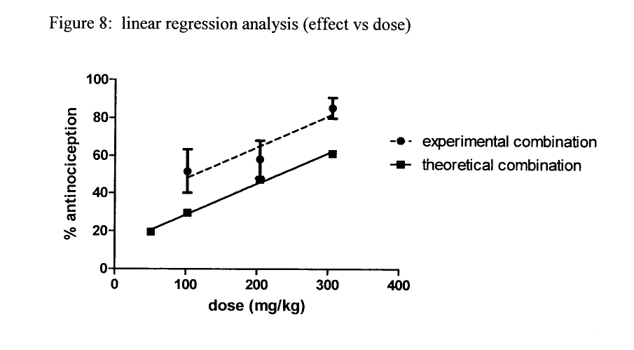

Doses and % antinociceptive activity data were analyzed for synergistic interactions using the PharmaTools Pro suite of programs (The McCary Group, Inc). Linear regression analysis (effect vs. log dose) and (effect vs dose) for hydrocodone, 3-hydroxyacetanilide, and the experimental combination along with the theoretical line representing the expected additive effect from the combination are shown in FIGS. 7 and 8, respectively.

The theoretical line of additive effect was calculated by analyzing the log linear regressions of the individual drugs and their maximum effects to give an additive effect for the dose pairs that had been tested experimentally. A comparison of the two regression lines (experimental vs theoretical) using the F-distribution to detect a difference in either the slope, the intercept(position), or both, for the lines returned a Calculated F=9.800 and Tabular F=9.550 suggesting that the regression lines differ significantly. A significant difference in the regressions can be interpreted as a departure from simple additivity.

Using the Computed Slopes and Y intercepts from the linear regression analysis: effect vs. log dose for hydrocodone and 3-hydroxyacetanlide the calculated interaction indecies are shown in Table 3 below. Interactions with calculated interaction indecies of <1 are considered to be synergistic. In this experiment 3 of the 4 dose combinations tested have interaction indecies less than 1.

TABLE 3
Results from interaction index calculations

| total dose | log dose | | |
|---|---|---|---|
| (mg/kg) | (mg/kg) | effect (% AA) | interaction index |
| 51 | 1.71 | 17.03 | 1.272 +/− 0.241 |
| 102 | 2.01 | 51.73 | 0.459 +/1 0.065 |
| 204 | 2.31 | 58.11 | 0.692 +/− 0.090 |
| 306 | 2.49 | 85.23 | 0.326 +/− 0.043 |

### EXAMPLE 2

One thousand tablets, each containing 350 mg of 3-hydroxyacetanilide and 10 mg hydrocodone bitartrate are prepared from the following types and amounts of ingredients: 3-hydroxyacetanilide micronized 350 gm, hydrocodone bitartrate 10 gm, lactose 75 gm, corn starch 50 gm, magnesium stearate 4 gm, and light liquid petrolatum 5 gm.

The 3-hydroxyacetanilide and hydrocodone bitartrate (finely divided by means of an air micronizer) are added to the other ingredients and then thoroughly mixed and slugged. The slugs are broken down by forcing then through a number sixteen screen. The resulting granules are then compressed into tablets, each tablet containing 350 mg of 3-hydroxyacetanilide and 10 mg of hydrocodone bitartrate.

Using the procedure above, tablets are similarly prepared containing hydrocodone bitartrate in 7.5 mg and 3.75 mg amounts by substituting 7.5 gm and 3.75 gm of hydrocodone bitartrate the 10 gm used above. These tablets are used to reduce the narcotic dose of the preceding example.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A pharmaceutical composition comprising an analgesic amount of an opioid analgesic or at least one pharmaceutically acceptable salt thereof, and 3-hydroxyacetanilide.

2. The pharmaceutical composition of claim 1 in an oral dosage form.

3. The pharmaceutical composition of claim 1, which contains, in addition a suitable pharmaceutical carrier or carriers.

4. The pharmaceutical composition of claim 1 wherein the opioid analgesic is selected from the group consisting of oxycodone or hydrocodone, or a pharmacologically acceptable salt, thereof.

5. The pharmaceutical composition of claim 1 wherein the opioid analgesic is codeine, or a pharmacologically acceptable salt, thereof.

6. The pharmaceutical composition of claim 1 wherein the opioid analgesic is hydrocodone, or a pharmacologically acceptable salt, thereof at a dose per 100 ml, which has no more than 300 mg of dissolved hydrocodone in addition to the therapeutic amount of 3-hydroxacetanilide.

7. The pharmaceutical composition of claim 1 wherein the opioid analgesic is hydrocodone, or a pharmacologically acceptable salt, thereof at a dose per dosage unit, which has no more than 15 mg of hydrocodone in addition to the therapeutic amount of 3-hydroxacetanilide.

8. The pharmaceutical composition of claim 1 wherein the opioid analgesic is oxycodone, or a pharmacologically acceptable salt, thereof at a dose per dosage unit, which has no more than 10 mg of oxycodone in addition to the therapeutic amount of 3-hydroxacetanilide.

9. The pharmaceutical composition of claim 1 wherein the opioid analgesic is codeine, or a pharmacologically acceptable salt, thereof, at a dose per dosage unit, which has no more than 60 mg of codeine in addition to the therapeutic amount of 3-hydroxacetanilide.

10. The pharmaceutical composition of claim 1 wherein the opioid analgesic is selected from the group consisting of a prodrug of oxycodone or a prodrug of hydrocodone, or a pharmacologically acceptable salt, thereof.

11. The pharmaceutical composition of claim 1 which comprises up to about 20 mg of hydrocodone, 3-hydroxyacetanilide, and a pharmaceutically acceptable carrier.

12. The pharmaceutical composition of claim 1 which comprises up to about 15 mg of hydrocodone, 3-hydroxyacetanilide, and a pharmaceutically acceptable carrier.

13. The pharmaceutical composition of claim 1 which comprises up to about 10 mg of hydrocodone, 3-hydroxyacetanilide, and a pharmaceutically acceptable carrier.

14-23. (canceled)

24. A method for treating pain in an animal, comprising administering an opioid analgesic or a pharmaceutically acceptable salt thereof to the animal, and orally administering 3-hydroxyacetanilide to the animal.

25. A method for treating pain in a human comprising administering an opioid analgesic or a pharmaceutically acceptable salt thereof, and 3-hydroxyacetanilide to the human.
